(19) United States
(12) Patent Application Publication
(10) Pub. No.: US 2026/0344976 A1
(43) Pub. Date: Nov. 5, 2026

(54) LOCKING MECHANISMS FOR ENDOSCOPIC DEVICES AND RELATED METHODS

(71) Applicant: Boston Scientific Scimed, Inc., Maple Grove, MN (US)

(72) Inventors: Douglas MELANSON, Natick, MA (US); Sean POWELL, Holden, MA (US); Kenneth R. KEENE, Winchester, MA (US)

(73) Assignee: Boston Scientific Scimed, Inc., Maple Grove, MN (US)

(21) Appl. No.: 19/657,363

(22) Filed: Apr. 24, 2026

Related U.S. Application Data
(60) Provisional application No. 63/797,219, filed on Apr. 30, 2025.

(51) Int. Cl. A61B 1/00

(52) U.S. Cl. CPC A61B 1/00042; A61B 1/00066; A61B 1/00098; A61B 1/00128

(57) ABSTRACT

A medical device includes a shaft and a handle. A distal end of the shaft includes an elevator. The handle includes an actuator and a locking mechanism. The actuator is operably connected to the elevator. The locking mechanism is configured to secure the actuator. The locking mechanism includes a contact portion on a portion of a handle body. The actuator is movable in a first direction and a second direction to control a position of the elevator, and at least a portion of the actuator is movable in a third direction and fourth direction such that at least a portion of the actuator is spaced away from the contact portion or at least partially overlaps with the contact portion. At least a portion of the actuator is pivotable to engage with and disengage from the contact portion to secure the position of the actuator relative to the handle body.

### CROSS-REFERENCE TO RELATED APPLICATIONS

This application claims priority to U.S. Provisional Application No. 63/797,219, filed on Apr. 30, 2025, which is incorporated by reference herein in its entirety.

### TECHNICAL FIELD

Various aspects of this disclosure relate generally to devices and methods for inhibiting movement of components of medical devices. In embodiments, the disclosure relates to devices for locking components of handles of duodenoscopes.

### BACKGROUND

Duodenoscopes may include a handle and a sheath, and the sheath may be insertable into a body lumen of a subject. The sheath may terminate in a distal tip portion, which may include features such as optical elements (e.g., camera, lighting), air/water outlets, and working channel openings. An elevator may be disposed at a distal tip and may be actuatable in order to change an orientation of a medical device/tool passed through the working channel. For example, the elevator may be pivotable or otherwise movable.

Elements in the handle may control the elements of the distal tip. For example, buttons, knobs, levers, etc. may control elements of the distal tip. The elevator may be controlled via a control mechanism in a handle, such as a lever, which may be attached to a control wire that attaches to the elevator. When an actuator (e.g., a lever) is actuated, the wire may move proximally and/or distally, thereby raising and/or lowering the elevator. In some aspects, the elevator may be movable between a plurality of or range of positions, and the user may need to maintain the position of the actuator in order to maintain the position of the elevator, which may stress, fatigue, or otherwise occupy the user.

### SUMMARY

Each of the aspects disclosed herein may include one or more of the features described in connection with any of the other disclosed aspects.

A medical device may include a shaft and a handle. The shaft may be configured to be inserted into a body lumen of a patient. A distal end of the shaft may include an elevator for changing an orientation of a medical instrument delivered through the shaft. The handle may include a handle body. The handle may include an actuator and a locking mechanism. The actuator may be operably connected to the elevator. Movement of the actuator may cause movement of the elevator. The actuator may be configured to be contacted by a user. The locking mechanism may be configured to secure a position of the actuator in one of a plurality of actuator positions relative to the handle body to secure a position of the elevator in a plurality of elevator positions. The locking mechanism may include a contact portion on a portion of the handle body. The actuator may be movable in a first direction and a second direction to control a position of the elevator. At least a portion of the actuator may be movable in a third direction and fourth direction such that at least a portion of the actuator is spaced away from the contact portion or at least partially overlaps with the contact portion. At least a portion of the actuator may be pivotable to engage with and disengage from the contact portion to secure the position of the actuator relative to the handle body.

The medical device may include one or more of the following features. The actuator may include a projection extending radially inward toward a surface of the handle body. The contact portion of the handle body may include a rack with a plurality of teeth configured to lockably engage with the projection on the actuator. The projection may include an engaging surface configured to engage with one or more portions of the rack on the handle body. At least one of the contact surface and the projection of the actuator may be movable in the third direction and the fourth direction, between a first configuration, in which the engaging surface is unaligned with the rack on the handle body, and a second configuration, in which the engaging surface is aligned with the rack on the handle body. The actuator may be pivotable in a fifth direction and a sixth direction. The fifth direction may be parallel to the first direction, and the sixth direction may be parallel to the second direction. Movement of the actuator in the fifth direction may cause the projection to engage with one or more teeth of the rack, and movement of the actuator in the sixth direction may cause the projection to disengage with the one or more teeth of the rack. The actuator may include an arm and a contact portion, and the contact portion may be laterally movable relative to the arm. The engaging surface of the projection may include a plurality of teeth. The third and fourth directions may be approximately perpendicular to each of the first direction and the second direction. A length of the rack may be longer than a length of the projection.

In one or more other aspects, the locking mechanism further may include a wire. A first end of the wire may be secured to a portion of the handle, and the actuator may include a plurality of openings configured to receive a second end of the wire. The handle may include at least one collet to secure the first end of the wire to the handle. The wire may be rigid. The actuator may include a plurality of openings arranged in a curved row. The actuator may include a lever.

In another aspect, a medical device may include a shaft and a handle. The shaft may be configured to be inserted into a body lumen of a patient. A distal end of the shaft includes an elevator for changing an orientation of a medical device. The handle may include a handle body and a lever. A portion of the handle body may include a track with a plurality of teeth. The lever may be operably connected to the elevator. Movement of the lever in first or second directions may cause movement of the elevator. The lever may include an arm and a contact portion. The contact portion may be configured to be contacted by a user. The contact portion may be pivotable and may include a projection extending toward the handle body. The projection may include an engaging surface. Pivoting of the contact portion relative to the arm may cause the engaging surface of the projection to contact the teeth of the rack to lock a position of the lever relative to the handle body.

The medical device may include one or more of the following features. At least a portion of the lever may be movable in a third direction and a fourth direction, approximately perpendicular to each of the first direction and the second direction, between a first configuration, in which the engaging surface is unaligned with the rack on the handle body, and a second configuration, in which the engaging surface is aligned with the rack on the handle body. The lever may be pivotable in a fifth direction and a sixth direction. The fifth direction may be parallel to the first direction. The sixth direction may be parallel to the second direction. Movement of the lever in the fifth direction may cause the projection to engage with one or more teeth of the rack, and movement of the lever in the sixth direction may cause the projection to disengage with the one or more teeth of the rack.

In yet another aspect, a medical device handle may include an actuator and a handle body. The actuator may be configured to be operably connected to an elevator, such that movement of the actuator is configured to cause movement of the elevator. The actuator may include a contact portion configured to be contacted by a user. The handle body may include an engaging surface adjacent to the actuator. The actuator may include a button on a portion of the actuator and a click mechanism internal to the actuator. The button may be depressible relative to the actuator. A first end of the button may extend from a first side of the actuator, and a second end of the button may extend from a second side of the actuator. The click mechanism may be configured to selectively retain the button in a depressed configuration. The second end of the button may be configured to engage with the engaging surface to at least partially secure a position of the actuator relative to the handle body.

The medical device handle may include one or more of the following features. The engaging surface may include a plurality of indentations. Each of the indentations may be configured to receive a portion of the second end of the button.

It may be understood that both the foregoing general description and the following detailed description are exemplary and explanatory only and are not restrictive of the invention, as claimed. As used herein, the terms “comprises,” “comprising,” “includes,” “including,” “has,” “having,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/- 10% of a stated value.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

### BRIEF DESCRIPTION OF THE DRAWINGS

The accompanying drawings, which are incorporated in and constitute a part of this specification, illustrate aspects of the present disclosure and together with the description, serve to explain the principles of the disclosure.

FIG. 1A illustrates an exemplary medical device, and FIG. 1B illustrates a side view of a portion of a handle of the exemplary medical device of FIG. 1A, according to aspects of this disclosure.

FIGS. 2A-2C illustrate various configurations of an actuator and a locking mechanism relative to the handle of the exemplary medical device of FIGS. 1A and 1B, according to aspects of this disclosure.

FIG. 3 illustrates another actuator and locking mechanism, according to aspects of this disclosure

FIGS. 4A and 4B illustrate various perspective views of yet another actuator and locking mechanism, according to aspects of this disclosure.

### DETAILED DESCRIPTION

Particular aspects of this disclosure are described in greater detail below. The terms and definitions provided herein control, if in conflict with terms and/or definitions incorporated by reference.

After activating or positioning an elevator using a control mechanism, it may be desirable for an operator to be able to retain the elevator in a desired position without maintaining contact on the control mechanism used to activate the elevator. Embodiments of this disclosure relate to locking mechanisms for inhibiting motion of an elevator control lever when the lever is in one or more predetermined positions. The locking mechanisms may, for example, lock the elevator lever so that the elevator is retained in a raised position, a partially raised position, etc. After the elevator lever is locked, a user may be able to remove a finger or thumb from the elevator lever, freeing the user to conduct other operations with that finger or thumb. Locking/retaining mechanisms may help to avoid fatigue by the user. The examples disclosed herein detail various structures to lock, retain, or otherwise at least partially secure an actuator (e.g., a lever) in a desired position. Although elevator levers are described herein, it will be appreciated that the disclosed levers may also be used for other types of controls (e.g., steering of a distal tip of the duodenoscope).

Further, although duodenoscopes are referenced herein for illustration purposes, it will be appreciated that this disclosure encompasses any suitable delivery device or medical device configured to allow an operator to access and view internal body anatomy of a subject (e.g., patient) or to deliver medical or treatment instruments, such as, for example, electrosurgical knives, electrodes, biopsy forceps, graspers, baskets, snares, probes, scissors, retrieval devices, lasers, and other tools, into the subject’s body. The medical devices herein may be inserted into a variety of body lumens and/or cavities, such as, for example, the urinary tract or gastrointestinal tract. It will be appreciated that, unless otherwise specified, bronchoscopes, endoscopes, ureteroscopes, gastroscopes, endoscopic ultrasonography (“EUS”) scopes, colonoscopes, laparoscopes, cystoscopes, aspiration scopes, sheaths, catheters, or any other suitable delivery device or medical device may be used in connection with the features described herein

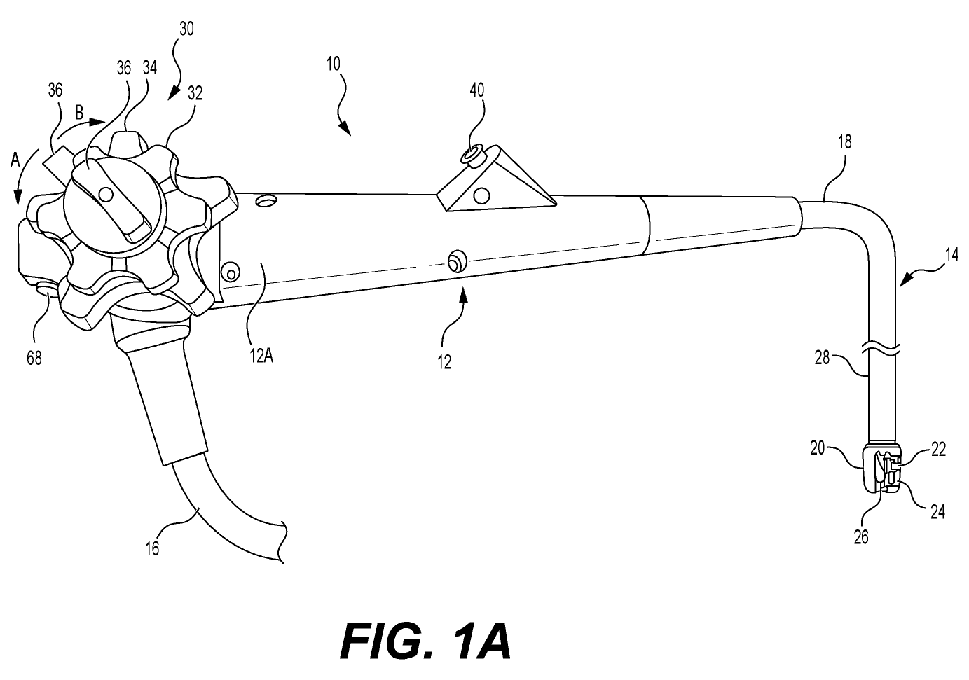

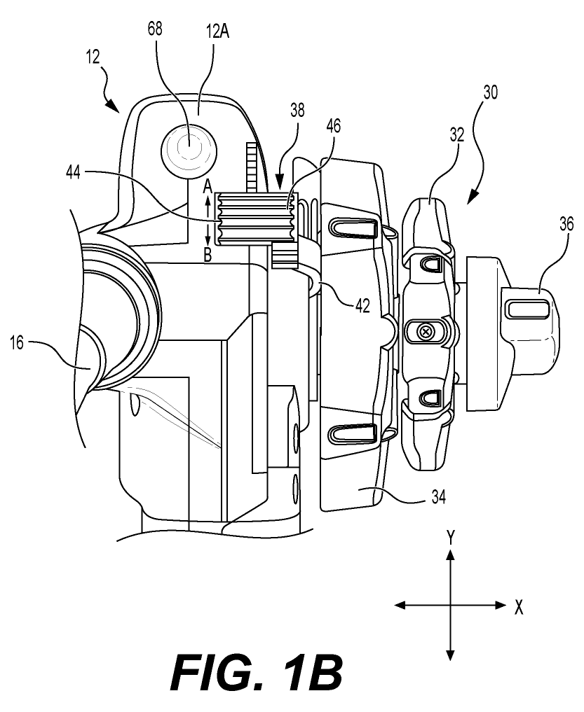

FIG. 1A depicts an exemplary insertion device, for example, a duodenoscope 10, including a handle 12 and an insertion portion 14. FIG. 1B shows a side view of a proximal portion of handle 12. Duodenoscope 10 may also include an umbilicus 16 for purposes of connecting duodenoscope 10 to sources of, for example, air, water, suction, power, etc., as well as to image processing and/or viewing equipment. Insertion portion 14 may include a sheath or shaft 18 and a distal tip 20. Distal tip 20 may include an elevator 26 for changing an orientation of a tool inserted in a working channel of duodenoscope 10. As shown in FIG. 1B, handle 12 includes an elevator control lever 38 that is movable to control a position of elevator 26. As discussed in detail below, elevator control lever 38 may be retained, locked, or otherwise secured via one or more mechanisms on handle 12, which also retains, locks, or otherwise secures the position of elevator 26.

Distal tip 20 may include an imaging device 22 (e.g., a camera) and a lighting source 24 (e.g., an LED or an optical fiber). Distal tip 20 may be side-facing. That is, imaging device 22 and lighting source 24 may face radially outward, perpendicularly, approximately perpendicularly, or otherwise transverse to a longitudinal axis of shaft 18 and distal tip 20. Additionally or alternatively, distal tip 20 may include one or more imaging devices 22 that face in more than one direction. For example, a first imaging device 22 may face radially outward, and a second imaging device 22 may face distally (approximately parallel to a longitudinal axis of distal tip 20/shaft 18)

Distal tip 20 may also include an elevator 26, for example, for changing the orientation of a tool inserted into a working channel of duodenoscope 10. Elevator 26 may alternatively be referred to as a swing stand, pivot stand, raising base, or any other suitable term. Elevator 26 may be pivotable via, for example, an actuation wire or another control element that extends from handle 12 (e.g., from elevator control lever 38), through shaft 18, to elevator 26

A distal portion of shaft 18 that is connected to distal tip 20 may have a steerable section 28. Steerable section 28 may be, for example, an articulation joint. Shaft 18 and steerable section 28 may include a variety of structures which are known or may become known in the art.

Handle 12 includes a handle body 12A and one or more actuators/control mechanisms 30. Control mechanisms 30 may provide control over steerable section 28 or may allow for provision of air, water, suction, etc. For example, handle 12 may include control knobs 32, 34 for left, right, up, and/or down control of steerable section 28. For example, one of knobs 32, 34 may provide left/right control of steerable section 28, and the other of knobs 32, 34 may provide up/down control of steerable section 28. Handle 12 may further include one or more locking mechanisms 36 (e.g., knobs or levers) for preventing steering and/or braking of steerable section 28 in at least one of an up, down, left, or right direction. As mentioned, handle 12 may include elevator control lever 38, and elevator control lever 38 may raise and/or lower elevator 26, via connection between lever 38 and an actuating wire that extends from lever 38, through shaft 18, to elevator 26. A port 40 (e.g., in handle 12) may allow passage of a tool through port 40, into a working channel of the duodenoscope 10, through shaft 18, to distal tip 20. Additionally, as shown in FIG. 1B, handle 12 may include one or more image or video capture buttons 68, which may be actuatable to control the capture of one or more images or video via imaging device 22. Handle 12 may also have one or more other features, such as one or more valves or one or more ports (e.g., for air, water, and/or suction).

In use, an operator may insert at least a portion of shaft 18 into a body lumen of a subject. Distal tip 20 may be navigated to a procedure site in the body lumen. The operator may insert a tool (not shown) into port 40, and pass the tool through shaft 18 via a working channel to distal tip 20. The tool may exit the working channel at distal tip 20. The user may use elevator control lever 38 to raise elevator 26 and angle the tool toward a desired location (e.g., a papilla of the pancreatico-biliary tract). The user may use the tool to perform a medical procedure

An elevator actuator, such as elevator control lever 38 may be moved in one or more directions to provide control over elevator 26. Although a lever 38 is depicted, alternative control mechanisms may fall under the scope of this disclosure. For example, sliders, knobs, switches, wheels, or other mechanisms may be used.

As shown in FIG. 1B, elevator control lever 38 may include an arm 42 and a contact portion 44, which may extend radially outward from arm 42 (to the left in FIG. 1B). Arm 42 may extend into handle 12, connecting to a wire or other structure (e.g., a shaft, strand, cable, etc.) that extends through handle 12 and shaft 18 and connects to elevator 26.

Contact portion 44 may extend approximately parallel to a surface of handle body 12A. Contact portion 44 may extend approximately perpendicularly to arm 42. Contact portion 44 may include one or more ridges 46 or other features to increase friction and or facilitate gripping between contact portion 44 and a user’s finger or thumb.

As discussed in detail below, movement (e.g., upward in direction A or downward in direction B relative to the orientation in FIG. 1B) of contact portion 44 may cause elevator 26 to raise or lower. Furthermore, additional movement (e.g., left, right, and/or pivoting forward or backward relative to the orientation in FIG. 1B) of contact portion 44 may lock or unlock the position of elevator control lever 38, and correspondingly help to lock or unlock the position of elevator 26.

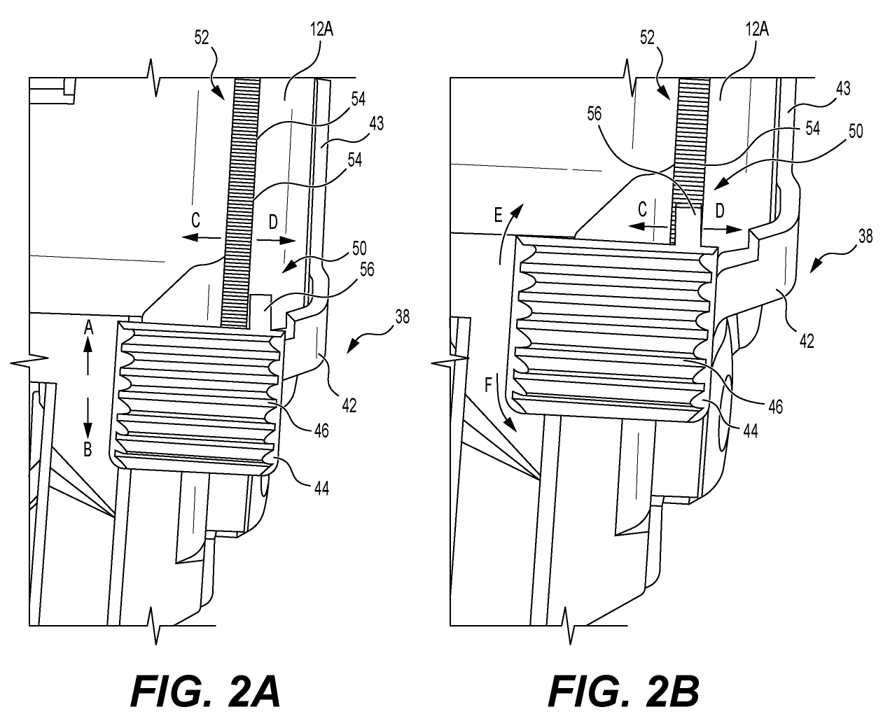

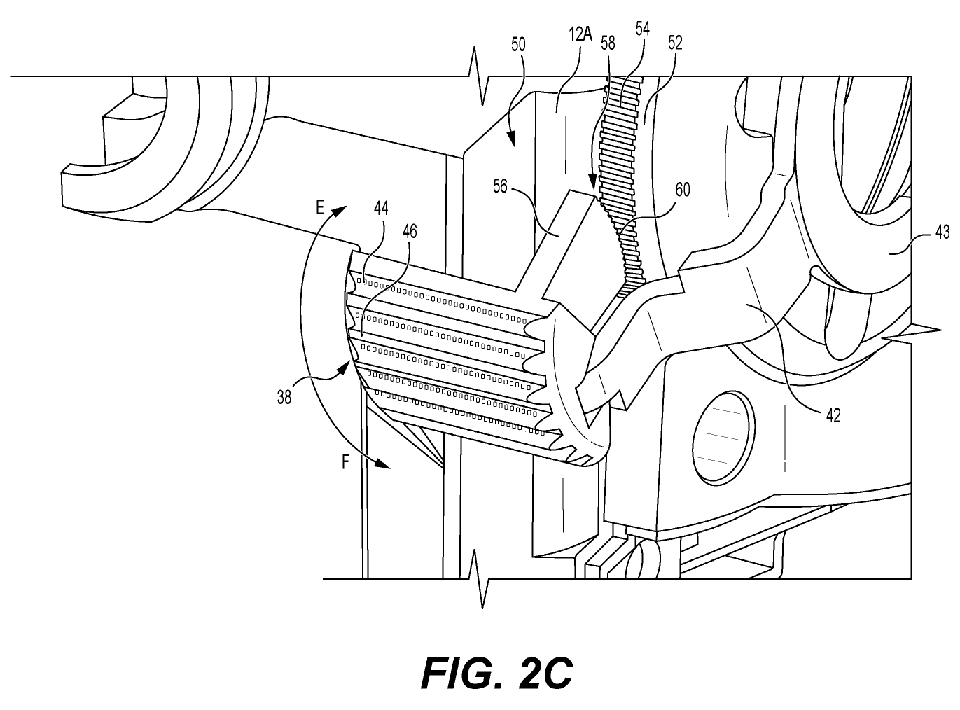

FIGS. 2A-4B depict various locking mechanisms. The locking mechanisms of FIGS. 2A-6B may be used along with handle 12 or with alternative handles. Aspects of the locking mechanisms described herein may be used alone or in combination. Where practical, like reference numbers have been used herein to denote similar structures. Unless stated otherwise, handles described below may have any of the features of handle 12. It will be appreciated that the various locking mechanisms may be used for locking the lever and the elevator at various positions. Although the locking mechanisms are described relative to an elevator lever and an elevator, it will be appreciated that the locking mechanisms may alternatively be used with other components (e.g., steering components).

FIGS. 2A-2C show a first exemplary locking mechanism 50. Locking mechanism 50 may include an actuator, such as lever 38 having arm 42 and contact portion 44. Arm 42 may be connected to a ring 43, which may rotate or pivot with the rotation of pivoting of contact portion 44. Ring 43 may be coupled to one or more wires, strings, strands, shafts, cables, etc. to impart movement to elevator 26 (FIG. 1A). In this example, handle body 12A includes a rack 52 with one or more teeth 54, and lever 38 is selectively lockable in various positions by engaging a portion of lever 38 with a portion of rack 52, as discussed below. For example, lever 38 may include a projection 56 (FIG. 1C), for example, extending in an opposite direction to contact portion 44 and toward handle body 12A and rack 52. Projection 56 may include an engaging surface 58 with a plurality of teeth 60 (FIG. 2C) configured to engage with one or more portions of rack 52. For example, lever 38 may be movable relative to handle body 12A such that engaging surface 58 of projection 56 may lockably engage with one or more portions of rack 52 on handle body 12A to help secure the position of lever 38 relative to handle body 12A, and thus help to secure the position, configuration, etc. of elevator 26. In these aspects, locking mechanism 50 may help to allow for lever 38 to be locked in any position through the stroke or movement range of lever 38.

FIG. 2A shows locking mechanism 50 in a first, unlocked configuration. Lever 38 is freely movable (subject to other constraints, such as a stop at the end of a range of lever 38). For example, lever 38 is moveable in direction A or in direction B, in order to control the position of elevator 26. In some aspects, moving lever 38 in direction A may include moving lever 38 upward (e.g., relative to the configuration shown in FIG. 1B), toward image capture button 68, etc. In some aspects, moving lever 38 in direction A may include moving lever 38 forward as the user is holding handle body 12A. In other aspects, moving lever 38 in direction B may include moving lever 38 downward (e.g., relative to the configuration shown in FIG. 1B), away from image capture button 68, etc. In some aspects, moving lever 38 in direction B may include moving lever 38 backward as the user is holding handle body 12A. Additionally, lever 38 is movable in direction C or in direction D in order to transition lever 38 into or out of alignment with rack 52, as shown in FIG. 2B.

Specifically, FIG. 2B shows locking mechanism 50 in a second configuration. As shown in FIG. 2B, projection 56 of lever 38 is at least partially aligned or overlapping with rack 52. For example, at least contact portion 44 (e.g., with projection 56) may be movable in direction C (e.g., away from arm 42, to the left relative to handle body 12A, toward contact portion 52, to the left relative to the configuration shown in FIGS. 2A-2C, etc.) from the configuration of FIG. 2A, such that projection 56 is at least partially aligned with or overlapping with rack 52. Contact portion 44 may be movable in direction D (opposite to direction Cc, e.g., toward arm 42, to the right relative to handle body 12A, away from contact portion 52, to the right relative to the configurations shown in FIGS. 2A-2C, etc.) from the configuration shown in FIG. 2B back to the configuration shown in FIG. 2A, such that projection 56 is not at least partially aligned with or overlapping with rack 52. In these aspects, contact portion 44 may be laterally movable or slidable relative to at least a portion of arm 42 (e.g., a proximal portion of arm 42). Additionally, as shown in FIG. 2C, at least a portion of lever 38, for example, contact portion 44 with projection 56, may be pivotable. In these aspects, contact portion 44 may be pivotable relative to arm 42, for example, in direction E or in direction F.

FIG. 2C shows a perspective view of locking mechanism 50 in the second configuration, with projection 56 at least partially overlapping with rack 52 (e.g., similar to the configuration of FIG. 2B). As mentioned above, projection 56 includes engaging surface 58, which can at least partially engage with one or more portions of rack 52, for example, with one or more of teeth 54. For example, engaging surface 58 may include one or more lever teeth 60, which may engage with teeth 54 of rack 52 to help secure the position of lever 38, and thus help to secure the position of elevator 26. As mentioned above, contact portion 44 of lever 38 may rock or pivot, for example, relative to arm 42. In these aspects, contact portion 44 can be rocked or pivoted in direction E (e.g., toward rack 52 or handle body 12A, into the page relative to the configuration shown in FIGS. 2A-2C, etc.) to help lever teeth 60 engage with teeth 54 of rack 52 to secure the position of lever 38. Additionally, contact portion 44 can be rocked or pivoted in direction F (e.g., away from rack 52 or handle body 12A, out of the page relative to the configuration shown in FIGS. 2A-2C, etc.) to help disengage lever teeth 60 from teeth 54 of rack 52. Then, contact portion 44 may be moved in direction D (FIGS. 2A and 2B), such that lever 38 is freely movable relative to handle body 12A, for example, to reposition elevator 26. The positioning and locking steps may be performed as many times as necessary for a medical procedure. Additionally, locking mechanism 50 may include one or more springs or biasing elements, for example, configured to bias one or more portions of lever 38 (e.g., contact portion 44) toward one or more directions, positions, configurations, etc. As shown in FIGS. 2A-2C, rack 54 may include a length (e.g., in the vertical direction of FIGS. 2A-2C) that is longer than a length of projection 56, and rack 54 may include a width (e.g., in the horizontal direction of FIGS. 2A-2C) that is slightly wider than a width of projection 56.

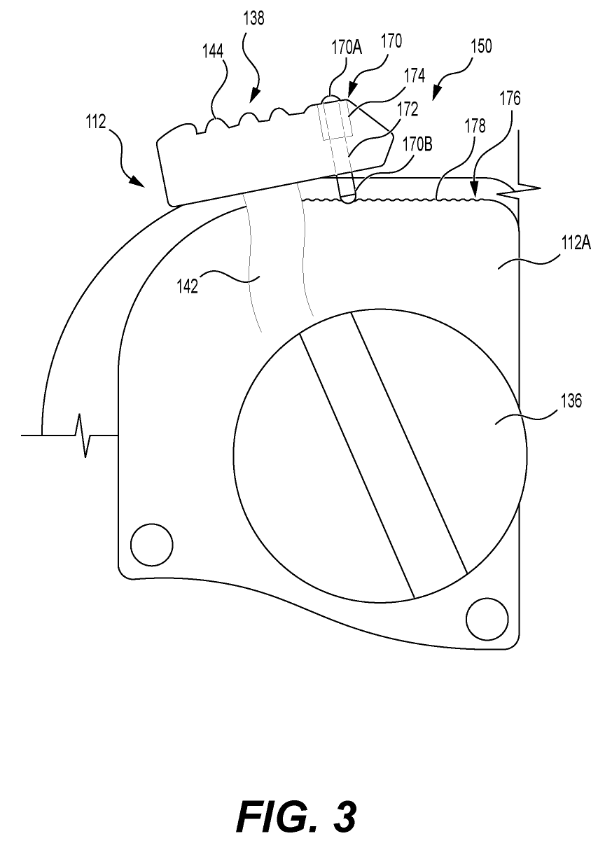

FIG. 3 shows an alternative locking mechanism 150. Locking mechanism 150 may make use of a pen-cap or click mechanism. Locking mechanism 150 includes an actuator, such as a lever 138, which may include an arm 142 and a contact portion 144. Lever 138 may be movable relative to a handle body 112A of a handle 112. Although not shown, handle 112 may include one or more knobs, for example, to control deflection of a shaft, and one or more locking mechanisms 136. As discussed in detail below, locking mechanism 150 may help to allow for lever 138 to be locked in any position through the stroke or movement range of lever 138. In some aspects, one or more features of the embodiment shown in FIG. 3 may be used in combination of features of other embodiments discussed here.

Lever 138 includes a pin, rod, or button 170, which may be movable through a channel or lumen 172 within contact portion 144 of lever 138. Button 170 includes a first end 170A, for example, at least partially extending from a first side of contact portion 144. Button 170 also includes a second end 170B, for example, at least partially extending from a second side of contact portion 144, opposite to the first side of contact portion 144, and configured to selectively contact a portion of handle body 112A. At least second end 170B of button 170 may be formed of or otherwise includes a polymeric material. Button 170 may be lockably movable relative to contact portion 144. For example, contact portion 144 may include a spring or selective click mechanism 174 to control the position of button 170 relative to contact portion 144.

As shown in FIG. 3, handle body 112A may include an engaging surface 176, for example, including a plurality of indentations 178, grooves, ridges, roughened surfaces, etc. In some aspects, second end 170B of button 170 may be at least partially received within one or more of indentations 178 to help secure the position of lever 138 relative to handle body 112A, and thus help to secure the position of elevator. For example, second end 170B may include one or more teeth, projections, etc., which may engage with one or more indentations 178. In these aspects, a user may position lever 138 in a desired position and then depress button 170 (e.g., via first end 170A). Click mechanism 174 may help to secure button 170 in the depressed configuration.

Additionally, in the depressed configuration, second end 170B of button 170 may at least partially engage with or abut a portion of handle body 112A to help secure the position of lever 138. For example, as mentioned above, second end 170B of button 170 may be at least partially received within one indentation 178. Nevertheless, in some aspects, handle body 112A may not include indentations 178, but the surface properties of handle body 112A at engaging surface 176 or the material properties of second end 170B of button 170 may help to secure the position of lever 138 relative to handle body 112A, and thus help to secure the position of an elevator. Then, the user may again depress or apply downward pressure to button 170 to release click mechanism 174, which allows button 170 to return to an unengaged position (e.g., such that second end 170B is spaced apart from engaging surface 176 of handle body 112A). For example, again depressing or applying pressure to button 170 may depress a spring or other mechanism within click mechanism 174, for example, without further moving button 170 (e.g., second end 170B) further toward handle body 112A, as second end 170B is engaged with or otherwise abutting handle body 112A. The user may then reposition lever 138 and the elevator. The positioning and locking steps may be performed as many times as necessary for a medical procedure.

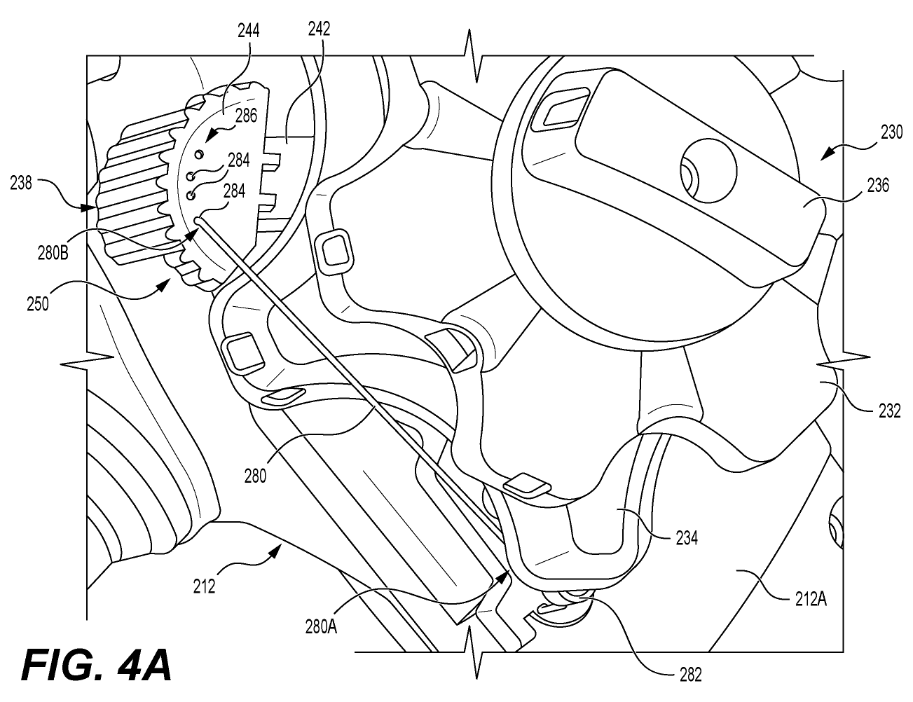

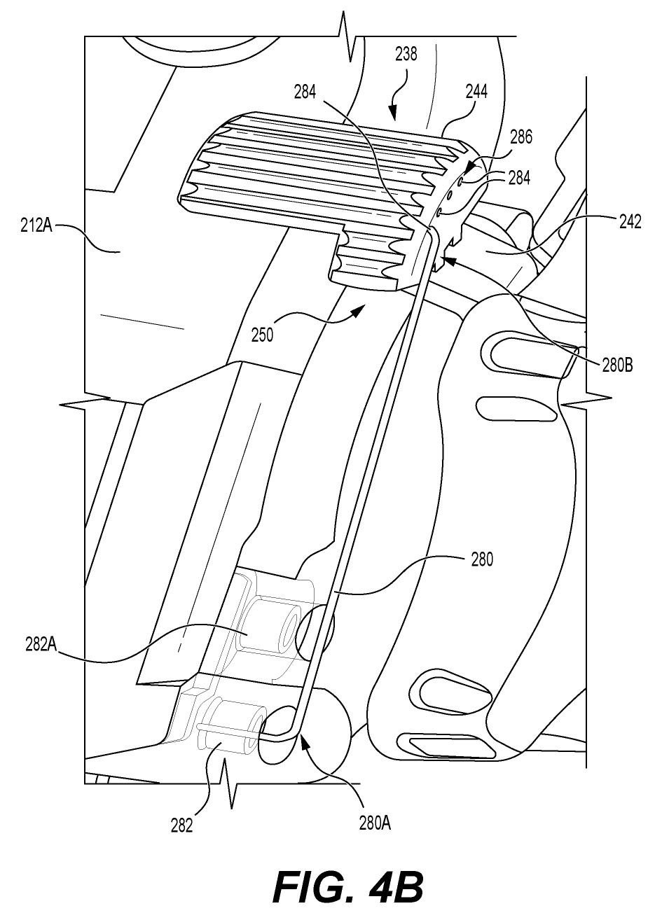

FIGS. 4A and 4B show another alternative locking mechanism 250. Locking mechanism 250 may make use of a wire or pin locking mechanism. Locking mechanism 250 includes an actuator, such as a lever 238, which may include an arm 242 and a contact portion 244, as discussed above. Lever 238 may be movable relative to a handle body 212A of a handle 212. As shown, handle 212 may include one or more control mechanisms 230 (e.g., knobs 232, 234), for example, to control deflection of a shaft, and one or more locking mechanisms 236. As discussed in detail below, locking mechanism 250 may help to allow for lever 238 to be locked in a plurality of positions through the stroke or movement range of lever 238. In some aspects, one or more features of the embodiment shown in FIGS. 4A and 4B may be used in combination of features of other embodiments discussed here.

Locking mechanism 250 includes a wire 280, or pin, bar, rope, cable, etc. Wire 280 may be at least partially rigid. A first end 280A of wire 280 is secured to a portion of handle body 212A, for example, via a collet 282, flange, socket, opening, hole, etc. For example, first end 280A of wire 280 may be wrapped around, welded, pinched, or otherwise secured to collet 282. As shown in FIG. 4B, handle body 212A may include one or more additional collets 282A, for example, to allow for different configurations of wire 280 and locking mechanism 250. In these aspects, first end 280A of wire 280 may include a bend or bent portion, such that first end 280A may be secured within one of collets 282, 282A.

A second end 280B of wire 280 may be selectively coupled to lever 238. For example, contact portion 244 of lever 238 includes a plurality of holes, slots, or other openings 284. For example, openings 284 may be arranged in an arced or curved configuration or row 286 on a lateral surface of contact portion 244, for example, a surface extending parallel to a surface of handle body 212A. As shown, contact portion 244 may include four openings 284. In other aspects, contact portion 244 may include other numbers of openings 284, for example, one opening 284, two openings 284, three openings 284, five or more openings 284. Additionally, openings 284 may be evenly or unevenly distributed along curved row 286. Openings 284 may be configured to receive at least a portion of second end 280B of wire 280, for example, to help secure the position of lever 238 relative to handle body 212A, and thus control the position of an elevator. In these aspects, second end 280B of wire 280 may include a bend or bent portion, such that second end 280B may be secured within one of openings 284.

For example, with lever 238 separated from wire 280, contact portion 244 of lever 238 may be manipulated to position lever 238 relative to handle body 212A, and thus to position the elevator. With lever 238 (and the elevator) in a desired position, a user may position second end 280B of wire 280 in one of openings 284. Because wire 280 is at least partially rigid, wire 280 may help to secure the position of lever 238 relative to handle body 212A, and thus help to secure the position of the elevator, without the need for the user to retain the position of lever 238 (e.g., with the user’s thumb). The user may uncouple second end 280B of wire 280 from lever 238 to reposition lever 238 relative to handle body 212A, and may then re-secure lever 238 relative to handle body 212B by positioning second end 280B of wire 280 in a different opening 284. The positioning and locking steps may be performed as many times as necessary for a medical procedure.

As discussed above, various aspects of this disclosure may allow for a user of a medical device to secure the position of a lever, and thus to secure the position of an elevator. As such, the user does not need to manually retain the position of the lever, for example, with the user’s thumb, finger, hand, etc., which may allow the user to use the thumb, finger, hand, etc. to perform other tasks or otherwise rest during the procedure. Additionally, various aspects of this disclosure may help to allow the user to secure the lever, and thus the elevator, in a plurality of different positions or configurations, for example, through a range of motion of the lever and the elevator. Accordingly, various aspects of this disclosure may help to reduce user fatigue, allow for the user to perform a procedure more quickly or more efficiently, or otherwise help in the performance of a medical procedure.

While principles of the present disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device, comprising:
a shaft configured to be inserted into a body lumen of a patient, wherein a distal end of the shaft includes an elevator for changing an orientation of a medical instrument delivered through the shaft; and
a handle having a handle body, wherein the handle includes:
an actuator, wherein the actuator is operably connected to the elevator, wherein movement of the actuator causes movement of the elevator, and wherein the actuator is configured to be contacted by a user; and
a locking mechanism, wherein the locking mechanism is configured to secure a position of the actuator in one of a plurality of actuator positions relative to the handle body to secure a position of the elevator in a plurality of elevator positions, wherein the locking mechanism includes a contact portion on a portion of the handle body,
wherein the actuator is movable in a first direction and a second direction to control a position of the elevator,
wherein at least a portion of the actuator is movable in a third direction and fourth direction such that at least a portion of the actuator is spaced away from the contact portion or at least partially overlaps with the contact portion, and
wherein at least a portion of the actuator is pivotable to engage with and disengage from the contact portion to secure the position of the actuator relative to the handle body.

2. The medical device of claim 1, wherein the actuator includes a projection extending radially inward toward a surface of the handle body.

3. The medical device of claim 2, wherein the contact portion of the handle body includes a rack with a plurality of teeth configured to lockably engage with the projection on the actuator.

4. The medical device of claim 3, wherein the projection includes an engaging surface configured to engage with one or more portions of the rack on the handle body.

5. The medical device of claim 4, wherein at least one of the contact surface and the projection of the actuator are movable in the third direction and the fourth direction, between a first configuration, in which the engaging surface is unaligned with the rack on the handle body, and a second configuration, in which the engaging surface is aligned with the rack on the handle body.

6. The medical device of claim 5, wherein the actuator is pivotable in a fifth direction and a sixth direction, wherein the fifth direction is parallel to the first direction, wherein the sixth direction is parallel to the second direction, wherein movement of the actuator in the fifth direction causes the projection to engage with one or more teeth of the rack, and wherein movement of the actuator in the sixth direction causes the projection to disengage with the one or more teeth of the rack.

7. The medical device of claim 6, wherein the actuator includes an arm and a contact portion, wherein the contact portion is laterally movable relative to the arm.

8. The medical device of claim 5, wherein the engaging surface of the projection includes a plurality of teeth.

9. The medical device of claim 5, wherein the third and fourth directions are approximately perpendicular to each of the first direction and the second direction.

10. The medical device of claim 5, wherein a length of the rack is longer than a length of the projection.

11. The medical device of claim 1, wherein the locking mechanism further includes a wire, wherein a first end of the wire is secured to a portion of the handle, and wherein the actuator includes a plurality of openings configured to receive a second end of the wire.

12. The medical device of claim 11, wherein the handle includes at least one collet to secure the first end of the wire to the handle.

13. The medical device of claim 11, wherein the wire is rigid.

14. The medical device of claim 11, wherein the actuator includes a plurality of openings arranged in a curved row.

15. The medical device of claim 1, wherein the actuator includes a lever.

16. A medical device, comprising:
a shaft configured to be inserted into a body lumen of a patient, wherein a distal end of the shaft includes an elevator for changing an orientation of a medical device; and
a handle, wherein the handle includes:
a handle body, wherein a portion of the handle body includes a rack with a plurality of teeth; and
a lever, wherein the lever is operably connected to the elevator, wherein movement of the lever in first or second directions causes movement of the elevator, wherein the lever includes an arm and a contact portion, wherein the contact portion is configured to be contacted by a user, wherein the contact portion is pivotable and includes a projection extending toward the handle body, and wherein the projection includes an engaging surface,
wherein pivoting of the contact portion relative to the arm causes the engaging surface of the projection to contact the teeth of the rack to lock a position of the lever relative to the handle body.

17. The medical device of claim 16, wherein at least a portion of the lever is movable in a third direction and a fourth direction, approximately perpendicular to each of the first direction and the second direction, between a first configuration, in which the engaging surface is unaligned with the rack on the handle body, and a second configuration, in which the engaging surface is aligned with the rack on the handle body.

18. The medical device of claim 17, wherein the lever is pivotable in a fifth direction and a sixth direction, wherein the fifth direction is parallel to the first direction, wherein the sixth direction is parallel to the second direction, wherein movement of the lever in the fifth direction causes the projection to engage with one or more teeth of the rack, and wherein movement of the lever in the sixth direction causes the projection to disengage with the one or more teeth of the rack.

19. A medical device handle, comprising:
an actuator, wherein the actuator is configured to be operably connected to an elevator, such that movement of the actuator is configured to cause movement of the elevator, and wherein the actuator includes a contact portion configured to be contacted by a user; and
a handle body, wherein the handle body includes an engaging surface adjacent to the actuator,
wherein the actuator includes a button on a portion of the actuator and a click mechanism internal to the actuator, wherein the button is depressible relative to the actuator, wherein a first end of the button extends from a first side of the actuator, wherein a second end of the button extends from a second side of the actuator, wherein the click mechanism is configured to selectively retain the button in a depressed configuration, and wherein the second end of the button is configured to engage with the engaging surface to at least partially secure a position of the actuator relative to the handle body.

20. The medical device handle of claim 19, wherein the engaging surface includes a plurality of indentations, each configured to receive a portion of the second end of the button.
